(19) United States
(12) Patent Application Publication
(10) Pub. No.: US 2023/0233305 A1
(43) Pub. Date: Jul. 27, 2023

(54) METHOD OF AMELIORATING OR TREATING INFLAMMATORY IN AN ORAL CAVITY

(71) Applicants: Chung-Wei Chen, Taichung City (TW); Min-Jen Chiang, Taichung City (TW)

(72) Inventors: Chung-Wei Chen, Taichung City (TW); Min-Jen Chiang, Taichung City (TW)

(21) Appl. No.: 17/737,525

(22) Filed: May 5, 2022

(30) Foreign Application Priority Data
Jan. 22, 2022 (TW) 111102772

(51) Int. Cl. A61D 7/00; A61K 31/593; A61K 9/00; A61D 5/00

(52) U.S. Cl. CPC A61D 7/00; A61K 31/593; A61K 9/0063; A61D 5/00

(57) ABSTRACT

The present invention provides the method of meliorating or treating inflammatory in an oral cavity of subjects, comprising steps of excising the inflamed oral soft tissue, and promoting autophagy in an area of wound by application of vitamin D to effectively recovering the wound.

### CROSS-REFERENCE TO RELATED APPLICATION

This application claims priority of Taiwan patent application No. 111102772, filed on Jan. 22, 2022, the content of which are incorporated herein in its entirety by reference.

### BACKGROUND OF THE INVENTION

### 1. Field of the Invention

The present invention relates to a method of ameliorating or treating inflammatory in an oral cavity, and more particularly to a method of ameliorating or treating inflammatory in an oral cavity through excising inflamed oral soft tissue and recovering a wound by application of vitamin D.

### 2. The Prior Art

Stomatitis, periodontal disease, and tooth resorption are common dental diseases in felines and canines. Stomatitis is a disease in which the soft tissues of the oral cavity, including the oral mucosa and gums, become inflamed. Such inflammatory diseases may cause severe pain in patients, and symptoms such as poor appetite, bad mental condition, drooling, and reluctance to open the mouth due to the pain. Accidental ingestion of corrosive irritants, renal failure, bacterial or viral infection, autoimmune disorders, environmental factors and other reasons may cause stomatitis.

Among stomatitis of feline and canine, feline chronic gingivostomatitis (FCGS) is one of oral mucositis, which is a stomatitis disease of unknown etiology caused by local or extensive chronic inflammation, redness, and even ulceration of the gingiva or oral mucosa. Therefore, at present, symptoms may only be controlled by administering steroids, antibiotics, pain relievers, immunosuppressants, lactoferrin, interferon and other medicine, or surgical operation such as half-mouth or full-mouth tooth extraction, and cannot effectively improve symptoms or completely cure the disease.

The therapeutic effects of such treatment would differ in individuals, and the long-term administration of the aforementioned medicine would cause drug tolerance, and excessively administered doses may instead cause additional oral diseases. About 20-30% of individuals undergoing surgery would be completely ineffective, or even if the symptoms are improved, oral cavity of the individuals could not be completely closed due to tooth extraction which cause the oral cavity cannot maintain sufficient moisture with saliva and such condition in the mouth for a long time would cause the recurrence of stomatitis. Therefore, it is necessary and urgent to develop treatment methods or medicine that can effectively improve stomatitis of felines and canines and are less likely to recur.

### SUMMARY OF THE INVENTION

The objective of the present invention is to provide a method of ameliorating or treating inflammatory in an oral cavity of a subject, comprising the steps of: (a) demarcating a boundary between an inflamed oral soft tissue and a healthy oral soft tissue by using a tissue coagulant; (b) excising the inflamed oral soft tissue; and (c) recovering a wound caused by the step (b) by applying a composition; wherein, the composition contains a vitamin D.

In one embodiment of the present invention, the method further comprises the step of excising the diseased wall of a periodontal pocket to remove a proliferating inflamed tissue after the step (b).

In the embodiment of the present invention, the method further comprises the step of lifting a healthy and live oral soft tissue from a donor site to the wound to repair an exposed alveolar bone after the step (b).

In the embodiment of the present invention, the method further comprises the step of tooth extraction after the step (b).

In other embodiment of the present invention, the subject is a feline and a canine.

In the embodiments of the present invention, the subject is given general anesthesia prior to the step (a).

In the embodiments of the present invention, the inflamed oral soft tissue is caused by an oral mucositis or a feline chronic gingivostomatitis.

In the embodiments of the present invention, the vitamin D is a fat-soluble vitamin D or a water-soluble vitamin D, and an effective amount of the vitamin D is twice a day with 50-300 ng/kg each time.

In the embodiments of the present invention, the vitamin D promotes autophagy of the subject.

The mucogingival replacement surgery (MGRS) of the present invention can effectively treat animal oral soft tissue diseases, and the administration of the vitamin D composition of the present invention after surgical operation can effectively enhance the autophagy of mucosal epithelial cells in the area with surgery to reduce inflammation symptoms by regulating innate immunity and acquired immunity, so as to inhibit inflammation, promote wound healing, and maintain quality of life and effectively improve the prognosis of the gingival mucosal replacement surgery of the present invention.

### BRIEF DESCRIPTION OF THE DRAWINGS

FIG. 1 shows the stomatitis disease activity index (SDAI) score of the domestic cats with chronic oral mucositis, which are postoperatively treated with the vitamin D composition of the present invention after the surgical operation.

FIG. 2 shows the concentration of vitamin D in the serum of the domestic cats with chronic oral mucositis, which are postoperatively treated with the vitamin D composition of the present invention after the surgical operation.

FIG. 3A shows the observation of the oral inflamed mucosal tissues of the domestic cats with chronic oral mucositis, which are postoperatively treated with the vitamin D composition of the present invention after the surgical operation, performed immunohistochemistry (IHC) with anti-VDR.

FIG. 3B shows the quantified intensity of VDR in FIG. 3A.

FIG. 3C shows the quantified frequency of VDR in FIG. 3A.

FIG. 4A shows the observation of the oral inflamed mucosal tissues of the domestic cats with chronic oral mucositis, which are postoperatively treated with the vitamin D composition of the present invention after the surgical operation, performed immunohistochemistry (IHC) with anti-LC3B.

FIG. 4B shows the quantified intensity of LC3B in FIG. 4A.

FIG. 4C shows the quantified frequency of LC3B in FIG. 4A.

### DETAILED DESCRIPTION OF THE PREFERRED EMBODIMENT

The embodiments of the present invention are further described with the drawings. The following embodiments are provided to illustrate the present invention and are not intended to limit the scope of the present invention. One with ordinary skill in the art may have modifications and refinements without departing from the spirit and scope of the present invention. The scope of the present invention is defined by the scope of the appended claims.

The data provides in the present invention represent approximated, experimental values that vary within a range of ±20%, preferably ±10%, and most preferably ±5%.

Statistical analysis is performed using Excel and SigmaPlot. Data are expressed as mean±standard deviation (SD), and the two-way ANOVA analysis is used to analyze whether the difference between each group is statistically significant.

Unless otherwise specified in the context, the terms “a”, “the”, and the like used herein (especially in claims) should be understood to include both singular and plural forms.

The term “treat” or “treatment” should not be understood as treating an individual until complete recovery, but as maintaining the disease progression or symptom of an individual to a substantially static level, increasing the recovery rate of an individual, improving the severity of a specific condition, or improving the quality of life of a patient.

The term “modulate” or “modulation” refers to positive regulation (including induction, stimulation, and enhancement) or negative regulation (including inhibition, and attenuation) to cause an individual toward a normal state of the physiological function.

The “subject” refers to a non-human mammal, especially a feline or canine.

The mucogingival replacement surgery (MGRS) of the present invention is a compound course of treatment for oral soft tissue diseases of animals, especially for the oral soft tissue diseases of feline or canine, such as feline chronic gingivostomatitis (FCGS). The main concept of the mucogingival replacement surgery of the present invention is to replace the swollen and inflamed gingiva and oral mucosal tissues with collagen and scar tissues in the oral cavity of patients, that is, removing the inflamed gingiva and re-growing the healthy gums to effectively treat the oral soft tissue diseases of the patients.

The compound course of treatment for the mucogingival replacement surgery (MGRS) of the present invention may be roughly divided into the following: (1) surgical operation: the treatment of the mucogingival replacement surgery (MGRS) of the present invention is mainly surgical operation, which may include gingivectomy, gingival plasty and graft surgery, flap surgery, dental laser excision and cautery, teeth grinding, removing broken roots with piezosurgery, and other surgical operation to remove inflamed and swollen soft tissues and irritants, so as to allow new healthy tissues to re-grow; (2) internal medicine: after the inflamed and swollen soft tissues are removed by the surgical operation, medicine with forms of oral, injection, and topical may be used to assist wound healing, prevent wound infection, maintain the quality of life of the patient, and improve the therapeutic effect of the mucogingival replacement surgery (MGRS) of the present invention, and the medicine used herein may include vitamins, antibiotics, antibacterials, pain relievers, and appetite enhancers; (3) oral health education: paying attention to health care of wounds after the surgical operation, cleaning of the gingiva and oral mucosa, brushing teeth, and other health care matters for maintaining good oral hygiene to promote wound healing and avoid infection, and therefore to improve the therapeutic effect of the mucogingival replacement surgery of the present invention.

In the present invention, after performing the above-mentioned (1) surgical operation, further treatment with a vitamin D composition of the present invention may inhibit the inflammatory response in the operating area and promote osseointegration and wound healing to effectively improve the prognosis of mucogingival replacement surgery (MGRS) of the present invention. The vitamin D composition of the present invention may contain fat-soluble or water-soluble vitamin D, and the administration dose may be 50-300 ng/kg, preferably 100-250 ng/kg, more preferably 150-200 ng/kg, more preferably 200 ng/kg.

The present invention is further described with the following embodiments. The embodiments are provided to illustrate the present invention, not to limit the scope of the present invention. The scope of the present invention is shown in the appended claims.

### Example 1

### The Treatment for Ameliorating or Treating Inflammatory in an Oral Cavity

In one embodiment of the present invention, a preferred treatment of the mucogingival replacement surgery of the present invention is described herein. First, since the mucogingival replacement surgery has to be performed under general anesthesia, before the mucogingival replacement surgery (MGRS), a complete blood cell and blood biochemical examination on patients is necessary to be performed to ensure that the physical conditions of the patients could withstand the anesthesia, and controllable risks of the anesthesia are excluded as much as possible.

After excluding the controllable risks of the anesthesia, the treatment is performed according to the following method. First, after the patient is general anesthetized, tissue coagulants and/or topical hemostatic agents (such as silver nitrate and epinephrine) are used to demarcate the boundary between inflamed and healthy tissues. A dental laser (such as a diode laser) is then used for excision and cautery to remove the inflamed tissues.

If there are no other lesions (which will be described later) other than inflammation and swelling after surgical removal of the soft inflamed tissues, antibiotics such as doxycycline, clindamycin, or augmentin are used to prevent wound infections. In the case that the patients cannot administer the aforementioned antibiotics, a broad-spectrum antibacterial agent such as chlorhexidine may be applied topically. The dosage of chlorhexidine may be 0.05-0.2%, preferably 0.08-0.12%.

After the surgery operation, the vitamin D composition of the present invention may be used to inhibit inflammation and promote wound healing. The dosage of vitamin D may be orally administrated with 50-300 ng/kg, preferably 100-250 ng/kg, more preferably 150-200 ng/kg, more preferably 200 ng/kg. The oral mucositis tissue near the oropharynx may cause difficulty in swallowing and loss of appetite due to postoperative pain, so the usage of pain relief during and after the surgery operation is necessary to pay attention to. If the appetite of the patients is affected, an appetite stimulant may be administrated.

If the patients have other lesions (such as granuloma, tissue hyperplasia, and infection) other than inflammation and swelling, additional treatment such as scraping the lesioned tissues may be used to improve the therapeutic effect of the mucogingival replacement surgery (MGRS) of the present invention. For example, if there is obvious tissue proliferation, gingivectomy and oral mucosal excision are required to remove the proliferating inflamed tissues; wherein, surgical instruments such as surgical blades, retraction knives, or diode lasers may be used for such treatment. If the alveolar bone is exposed in the oral cavity or cannot be covered with the soft tissues, flap surgery or free gingival graft (FGG, a type of gingival plasty and graft surgery) is required to repair the exposed alveolar bone; wherein, surgical instruments such as surgical blades, periosteal strippers may be used for such treatment.

The inflamed oral mucosal tissue not near the gingival sulcus treated several times with the aforementioned manner to completely replace the inflamed tissues with collagen and scar tissues. If the responses are good and there are no other lesions, the swelling would gradually subside and heal. The inflamed and swelling oral mucosal tissues near the gingival sulcus are mostly juvenile perodontitis, and the retention of tooth may affect the therapeutic effect of the treatment. Therefore, after the patients are treated several times with the aforementioned manner, if the responses are not good, tooth extraction is required, but the priority is still to preserve the teeth.

### Example 2

The Treatment for Improving the Recovery after Surgical Operation by Vitamin D

In one embodiment of the present invention, in order to further understand the effects of the administration of vitamin D on suppressing inflammation and promoting wound healing after the mucogingival replacement surgery (MGRS), 18 domestic cats with feline chronic gingivostomatitis (FCGS) are recruited with the following conditions: the age is at least 1 year old, and the inflammatory response of the oral mucosa must last for at least 4 weeks; physical examination, complete blood count (CBC) examination, and blood biochemical profile examination should be completed within 2 weeks before sampling, and no obvious abnormality is found; feline immunodeficiency virus (FIV) and feline leukemia virus (FeLV) screening tests must be negative; the subject could to be orally fed with medicine by the owner.

Next, after the surgical operation is performed to remove the lesions in soft tissues by the treatment of Example 1, the subjects are randomly divided into the following three groups for testing: (A) the control group (n=6): the subjects are administered with only 5 mg/kg of deoxytetracycline for 6 weeks; (B) the experimental group (n=6): the subjects are administered with 5 mg/kg of deoxytetracycline+fat-soluble vitamin D for 6 weeks, wherein the fat-soluble vitamin D is orally administered with 200 ng/kg; and (C) the experimental group (n=6): the subjects are administered with 5 mg/kg of deoxytetracycline+water-soluble vitamin D for 6 weeks, wherein the water-soluble vitamin D is orally administered with 200 ng/kg. The fat-soluble and water-soluble vitamin D may be a liquid oral preparation, and deoxytetracycline may be mixed with the fat-soluble or water-soluble vitamin D to directly orally administered or packaged in a capsule for oral administration. At the 0th, 2nd, 4th, and 6th weeks of the test, the vitamin D concentration in the blood of the subjects is analyzed, and the stomatitis disease activity index (SDAI) is also evaluated. At the 0th and 6th weeks of the test, the inflamed oral mucosal tissues of the subjects from each group are collected by surgical scissors and surgical blades, and the collected tissues are immersed in 10% formalin for at least 24 hours and then embedded in paraffin Immunohistochemistry (IHC) is performed to observe the pathology of the tissues, as well as the content of VDR and LC3B in the tissues.

The stomatitis disease activity index is to score the condition of the subject by the following three aspects respectively and then add three scores to get the index: (1) owner evaluation (total score 0-3): the quality of life including appetite, activity level, grooming behavior, and perceived comfort would be scored by the owner, with a scale of 0-3, 0 is the best, and 3 is the worst; (2) weight (total score 0-3): the change in weight compared to most recent visit, with a scale of 0-3, 0 is an increase of more than 0.5 kg, 1 is an increase of 0.25-0.5 kg, 2 is an increase of 0-0.25 kg, and 3 is weight loss; (3) inflammatory symptoms (total score 0-24): inflammation in 8 different areas of the oral cavity, including maxillary buccal mucosa, mandibular buccal mucosa, maxillary attached gingiva, mandibular attached gingiva, lateral to palatoglossal folds, molar salivary gland, oropharynx, and sublingual area, with scale of 0-3, 0 is none, 1 is mild, 2 is moderate, and 3 is severe.

The procedure of collecting blood from the subjects in each group and obtaining serum thereof are briefly described as following: a 23 G injection needle with a 5 mL sterilized plastic syringe are used to collect about 4-6 mL blood from the jugular vein, cephalic vein, or medial saphenous vein of the subjects. The collected blood is then centrifuged at 10,000 rpm for 5 minutes (ARA mini 10) to separate serum and blood cells, and a new 5 mL sterilized syringe is used to collect the separated serum, which may be stored at −70° C. for subsequent testing.

After obtaining the serum of the subjects in each group, the serum samples stored at −70° C. is thawed at room temperature. The concentration of 25-(OH)-D3 in each serum sample is analyzed by electrochemiluminescence immunoassay (ECLIA) with Cobas E411 Rack detector (Roche) and Elecsys® Vitamin D total II detection kit (Roche). The unit is expressed in ng/mL. 25-(OH)-D3 is the stable form and main vitamin D component in the blood.

The procedure for analyzing the amount of vitamin D in the serum is briefly described as following: 20 μL of serum sample is mixed with reagent II (1 g/L dithiothreitol, pH 5.5) and reagent PT2 (28 g/L sodium hydroxide) of the aforementioned kits for the first reaction to release 25-(OH)-D3 from vitamin D-binding protein (VDBP). Reagent R1 (VDBP-Ru/(bpy)32+, containing: 100 μL VDBP labeled with staples; 100 mmol/L bis-tris propane buffer; 40 h/L albumin (human), pH 6.4; and preservative) is added for the second reaction to generate a complex. Reagent M (0.72 mg/mL micro-particles coated with streptavidin) and reagent R2 (140 ng/L 25-(OH)-D-biotin, 100 mmol/L bis-tris propane buffer, pH 8.6) are added for the third reaction to combine with the complex to form a reaction mixture. The reaction mixture is drawn into the measuring chamber, magnetically attracted to the electrode surface, and removed un-attracted substances with ProCell/ProCell M. A voltage is applied by an electrode to induce chemiluminescence, and a photomultiplier is used to detect the generated chemiluminescence. The master curve provided by the calibration curve, reagent barcode, or electronic barcode (e-barcode) generated exclusively by the two-point calibration instrument is used to obtain the analyzing results.

In the embodiment of the present invention, anti-vitamin D receptor (VDR) antibody and anti-microtubule associated protein 1 light chain 3B (MAP1LC3B, LC3B) antibody are used as primary antibodies for immunohistochemistry to observe the amount of vitamin D and autophagosome in the tissues. The procedure of immunohistochemistry is briefly described as following. All steps are performed on the Leica Bond Max automatic immunohistochemistry machine. The aforementioned inflamed oral mucosal tissues fixed with formalin and embedded in paraffin is sectioned with a thickness of 5 μm, and placed on a glass slide suitable for performing immunohistochemistry. The tissue section is placed in a 50° C. oven for 15 minutes and is dewaxed twice with Bond Dewax Solution for 5 minutes. The tissue section is washed three times with Tris buffered saline (TBS, pH 7.6) for 5 minutes, and then treated with Bond Leica Alcohol for 5 minutes for tissue rehydration. After being washed three times with TBS for 5 minutes, the tissue section is heated at 100° C. with Bond Citrate buffer (pH 6), and the tissue used for detecting VDR is heated for 20 minutes, and the tissue used for detecting LC3B is heated for 40 minutes. After being washed three times with TBS for 5 minutes, the tissue section is reacted with Bond Peroxide solution for 8 minutes. After being washed three times with TBS for 5 minutes, the tissue section is reacted with Bond Protein blocking solution for 10 minutes. After being washed three times with TBS for 5 minutes, the tissue section is reacted with the primary antibodies for 30 minutes at room temperature, wherein the dilution ratio of anti-VDR antibody (ab3508) is 500×, and the dilution ratio of anti-LC3B antibody (ab239416) is 50×. After being washed three times with TBS for 5 minutes, the tissue section is reacted with Bond Post primary solution containing rabbit anti-mouse IgG antibody for 10 minutes. After being washed three times with TBS for 5 minutes, the tissue section is reacted with Bond Post primary solution containing Poly-HPR IgG for 10 minutes. After being washed three times with TBS for 5 minutes, the tissue section is reacted twice with Bond DAB solution for 3 minutes for coloration. After being washed three times with TBS for 5 minutes, Bond Hematoxylin solution is used for staining the background for 10 minutes. The tissue section is washed three times with deionized water for 5 minutes, and then dried and mounted.

After the immunohistochemistry (IHC), the inflamed oral mucosal tissue is observed under an optical microscope at 100× and 400×. The staining intensity and frequency of oral mucosal epithelial cells in the tissues are evaluated by the method provided by Shackelford. The standards are as following: (1) staining intensity: 0 representing negative, no staining cells; 1 representing very faint staining; 2 representing mild staining; 3 representing mild to moderate staining; 4 representing moderate staining; 5 representing strong staining; and (2) staining frequency: 0 representing negative, no staining cells; 1 representing less than 5% cells staining; 2 representing 5-25% cells staining; 3 representing 26-50% cells staining; 4 representing 51-75% cells staining; 5 representing 76-100% cell staining.

VDR is a vitamin D receptor on cells, and the expression thereof may reflect the total amount of vitamin D acting on the cells. That is, the higher the expression levels of VDR, the greater the amount of vitamin D acting on the cells. LC3B is a subunit of microtubule associated protein 1A (MAP1A) and microtubule associated protein 1B (MAP1B), and plays a central role in the structure of the autophagosome membrane, so LC3B is often used as a biomarkers of autophagosomes.

The stomatitis disease activity index of the domestic cats with chronic oral mucositis, which are postoperatively treated with the vitamin D composition of the present invention after the surgical operation, is shown in FIG. 1. The cats in the control group (A) obtained scores of 11.44, 9.19, and 11.22 at the 0th, 2nd, and 6th weeks, respectively. The cats in the experimental group (B) administered with fat-soluble vitamin D obtained a score of 13 at the 0th week and a decreased score of 11.5 at the 6th week. The cats in the experimental group (C) administered with water-soluble vitamin D obtained a score of 17.67 at the 0th week and a decreased score of 13.92 at the 6th week. The results indicate that administration of the vitamin D composition of the present invention during the treatment of the gingival mucosal replacement surgery can effectively inhibit inflammation, promote wound healing, and maintain quality of life, so as to effectively improve the prognosis of the gingival mucosal replacement surgery of the present invention.

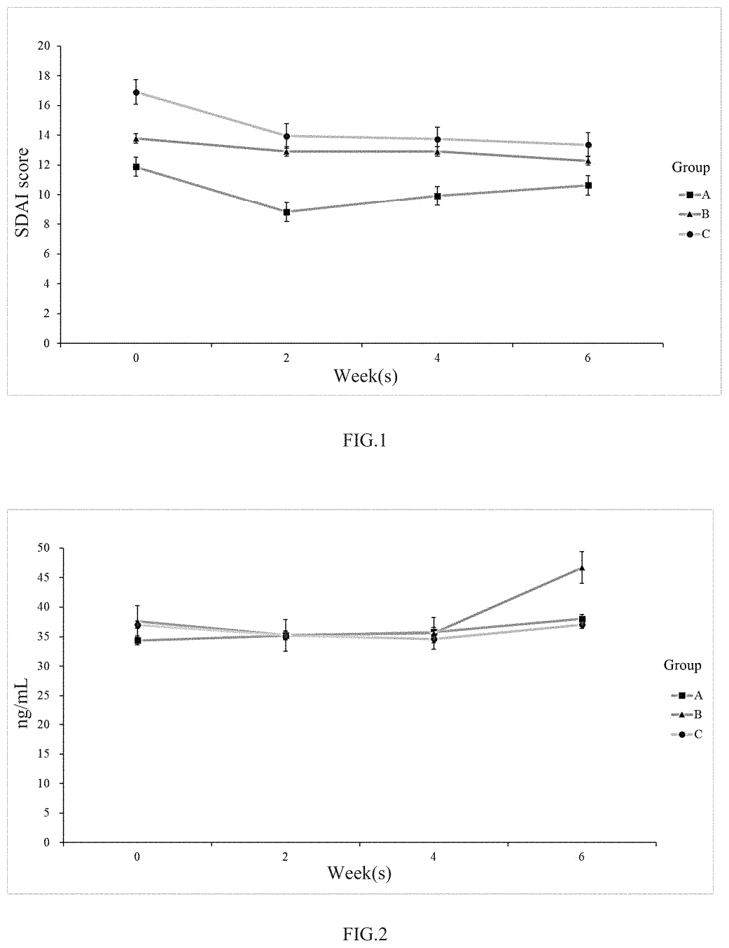

The concentration of vitamin D in the serum of the domestic cats with chronic oral mucositis, which are postoperatively treated with the vitamin D composition of the present invention after the surgical operation, is shown in FIG. 2. The amount of vitamin D in the serum of the cats in the three groups is between 30-40 ng/mL from the 0th to 6th weeks. During the period, the amount of vitamin D in the serum of the cats in the experimental group (B) administered with fat-soluble vitamin D is the highest. The results indicate that fat-soluble vitamin D may be more easily absorbed and utilized by the oral mucosal epithelial cells of the cats.

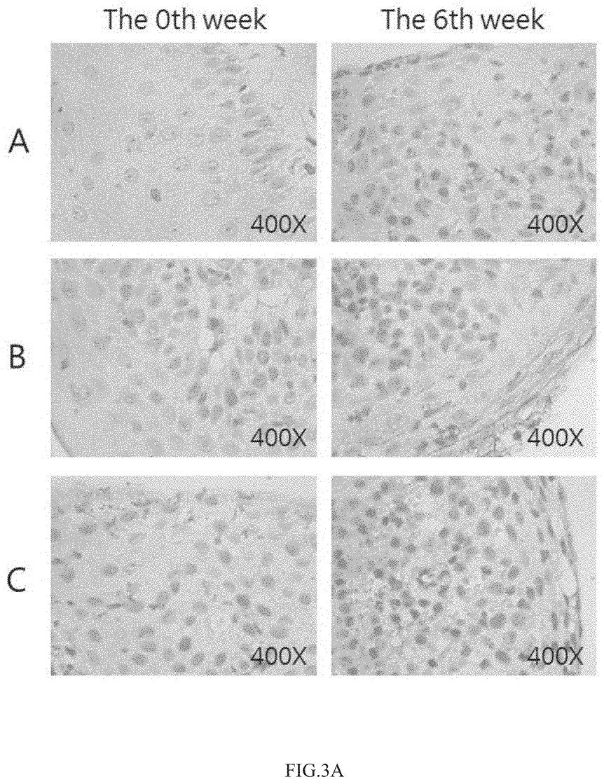

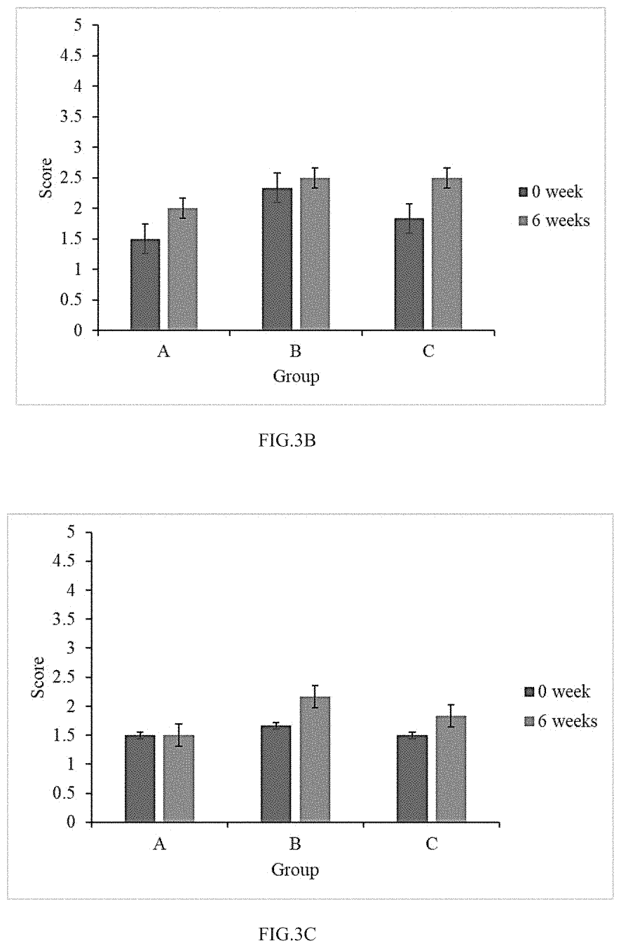

The observation of the oral inflamed mucosal tissues of the domestic cats with chronic oral mucositis, which are postoperatively treated with the vitamin D composition of the present invention after the surgical operation, performed immunohistochemistry with anti-VDR is shown in FIG. 3A. The intensity and frequency of VDR are shown in FIGS. 3B and 3C, respectively. VDR in the inflamed oral mucosal tissue increases only in the cats administered with fat-soluble vitamin D after 6-week treatment, while VDR decreases in the control group, and VDR remains unchanged in the cats administered with water-soluble vitamin D. The results also indicate that fat-soluble vitamin D is more easily absorbed by the oral mucosal epithelial cells of the cats.

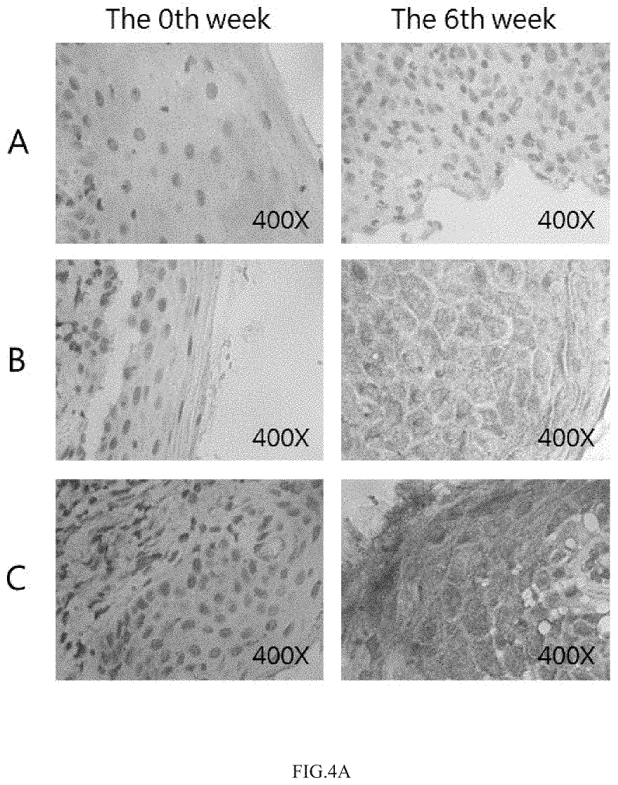

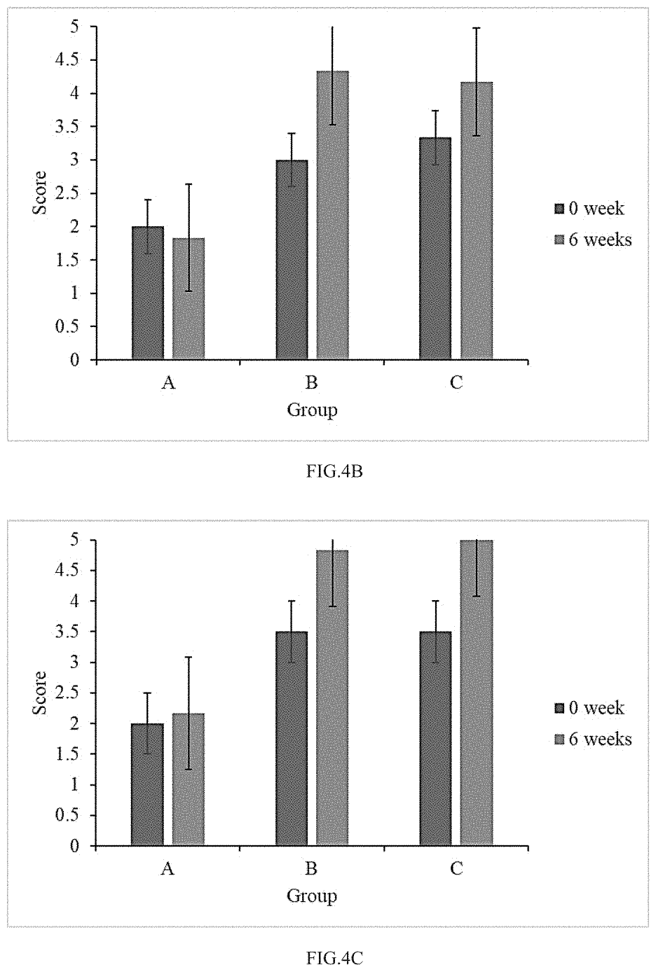

The observation of the oral inflamed mucosal tissues of the domestic cats with chronic oral mucositis, which are postoperatively treated with the vitamin D composition of the present invention after the surgical operation, performed immunohistochemistry with anti-LC3B is shown in FIG. 4A. The intensity and frequency of LC3B are shown in FIGS. 4B and 4C, respectively. LC3B in the inflamed oral mucosal tissue increases both in the cats administered with fat-soluble vitamin D and water-soluble vitamin D after 6-week treatment, while LC3B basically maintains a constant value in the control group. The results indicate that administration of the vitamin D composition of the present invention during the treatment of the gingival mucosal replacement surgery can effectively enhance the autophagy of mucosal epithelial cells in the area with surgery to improve the prognosis of the mucogingival replacement surgery of the present invention by regulating innate immunity and acquired immunity to reduce inflammation symptoms.

Together all the results of the embodiments of the present invention, the expression levels of VDR and LC3B in the oral mucosa of patients would increase, and the SDAI score would not increase when the administration of the vitamin D composition of the present invention after the surgical operation, indicating that the vitamin D composition of the present invention can effectively promote autophagy and maintain the effects of the surgical operation.

In summary, the mucogingival replacement surgery (MGRS) of the present invention can effectively treat animal oral soft tissue diseases, and the administration of the vitamin D composition of the present invention after surgical operation can effectively enhance the autophagy of mucosal epithelial cells in the area with surgery to reduce inflammation symptoms by regulating innate immunity and acquired immunity, so as to inhibit inflammation, promote wound healing, and maintain quality of life and effectively improve the prognosis of the gingival mucosal replacement surgery of the present invention.

What is claimed is:

1. A method of ameliorating or treating inflammatory in an oral cavity of a subject, comprising the steps of:
(a) demarcating a boundary between an inflamed oral soft tissue and a healthy oral soft tissue by using a tissue coagulant;
(b) excising the inflamed oral soft tissue; and
(c) recovering a wound caused by the step (b) by applying a composition;
wherein the composition contains a vitamin D.

2. The method according to claim 1, further comprising the step of excising the diseased wall of a periodontal pocket to remove a proliferating inflamed tissue after the step (b).

3. The method according to claim 1, further comprising the step of lifting a healthy and live tissue from a donor site to the wound to repair an exposed alveolar bone after the step (b).

4. The method according to claim 1, further comprising the step of tooth extraction after the step (b).

5. The method according to claim 1, wherein the subject is a feline and a canine.

6. The method according to claim 1, wherein the subject is given general anesthesia prior to the step (a).

7. The method according to claim 1, wherein the inflamed oral soft tissue is caused by an oral mucositis or a feline chronic gingivostomatitis.

8. The method according to claim 1, wherein the vitamin D is a fat-soluble vitamin D or a water-soluble vitamin D.

9. The method according to claim 1, wherein an effective amount of the vitamin D is twice a day with 50-300 ng/kg each time.

10. The method according to claim 1, wherein the vitamin D promotes autophagy of the subject.
